(19) United States
(12) Patent Application Publication
(10) Pub. No.: US 2021/0062157 A1
(43) Pub. Date: Mar. 4, 2021

(54) METHODS AND COMPOSITIONS FOR CULTURING ALVEOLAR CELLS

(71) Applicant: United Therapeutics Corporation, Silver Spring, MD (US)

(72) Inventors: Laila Roudsari, Durham, NC (US); Daren Robinson, Raleigh, NC (US); Benjamin Rusche, Durham, NC (US); Sajini Nair, Cary, NC (US); Ashley R. Dockham, Raleigh, NC (US)

(73) Assignee: United Therapeutics Corporation, Silver Spring, MD (US)

(21) Appl. No.: 17/003,431

(22) Filed: Aug. 26, 2020

Related U.S. Application Data
(60) Provisional application No. 62/892,206, filed on Aug. 27, 2019.

(51) Int. Cl. C12N 5/071

(52) U.S. Cl. CPC C12N 5/0688; C12N 2513/00; C12N 2527/00; C12N 2501/117; C12N 2501/415; C12N 2501/727; C12N 2501/11; C12N 2501/15

(57) ABSTRACT

Described herein are methods and compositions for use in expanding alveolar epithelial cells. The methods may include the use of three-dimensional substrates and improved techniques for expansion of the cells. The improved composition for culturing alveolar epithelial cells may include at least one or more of the following: a TGF-β pathway inhibitor; a Wnt pathway activator; a ROCK inhibitor; an epidermal growth factor (EGF); a keratinocyte growth factor (KGF); and a fetal bovine serum.

### CROSS REFERENCE TO RELATED APPLICATIONS

This application claims priority under 35 U.S.C. § 119(e) to U.S. Provisional Patent Application No. 62/892,206, filed Aug. 27, 2019, the content of which is hereby incorporated by reference in its entirety.

### TECHNICAL FIELD

This application relates generally to cell culture and more particularly, but without limitation, to methods and compositions for culturing alveolar cells, as well as cells produced from such methods.

### BACKGROUND

Expansion of alveolar epithelial cells is a significant challenge and a common roadblock in regenerative medicine. The alveolar epithelium is comprised of alveolar type 1 (AT1) cells, which cover 95% of lung epithelial surface area and are responsible for gas exchange, and alveolar type 2 (AT2) cells, which make up the remainder of the distal epithelium and produce surfactant. AT1 cells are thin, fragile cells, that are difficult to isolate and expand. In contrast, AT2 cells proliferate in response to lung injury in vivo and transdifferentiate into AT1 cells to repopulate damaged lung. As a result, expansion of AT2 cells is more readily achievable. However, in standard in vitro 2D culture conditions, human AT2 cells exhibit minimal proliferation and rapid loss of AT2 cell phenotype, as they transdifferentiate into non-proliferative AT1-like cells. In addition, AT2 cultures are often overgrown by a small starting population of contaminating airway basal cells or stromal cells. Further, studies have shown that AT2 cells may proliferate in organoid cultures (e.g., in Matrigel discs); however, such cultures are not scalable. Thus, there exists an unmet need for cell cultures and methods that assist in promoting successful expansion of alveolar epithelial cells.

### SUMMARY

The present disclosure addresses drawbacks of previously-known approaches by providing a method and composition for culturing alveolar epithelial cells on three-dimensional substrates. Three-dimensional culture confers certain advantages over standard two-dimensional culture of alveolar epithelial cells, as evidenced by improved maintenance of alveolar epithelial cell function and reduced overgrowth of contaminating airway basal cells.

A method for producing alveolar epithelial cells preferably includes preparing a plurality of three-dimensional substrates in a cell culture vessel, seeding a plurality of alveolar epithelial cells by combining the three-dimensional substrate and the alveolar epithelial cells in the cell culture vessel and providing conditions suitable to enable attachment of the cells to the three-dimensional substrate to create a suspension culture, promoting growth of the alveolar epithelial cells on or within the three-dimensional substrates, monitoring the culture for cell proliferation, and harvesting a plurality of alveolar epithelial cells from the three-dimensional substrates. In some embodiments, the alveolar epithelial cells may be AT2 cells. In certain other embodiments, the AT2 cells may be human alveolar type II epithelial cells.

The three-dimensional substrates can be at least one of a solid, microporous, or macroporous three-dimensional substrates. In certain embodiments, the three-dimensional substrate comprises a plurality of microcarriers. In certain embodiments, the substrate may be a macroporous, gelatin microcarrier. In some embodiments, the culture vessel may be a bioreactor or any vessel of similar volumetric dimensions. In some embodiments, the cell culture vessel may be a spinner flask. The three-dimensional substrates may also be present in a concentration of about 1-10 mg/mL of culture medium, e.g., 1-8, 1-6, 1-4, or 1-2 milligram of substrate per milliliter of culture medium.

Seeding may also include adding alveolar epithelial cells (e.g., either freshly isolated or cryopreserved) to culture media in the cell culture vessel. In some embodiments, seeding may include agitating the culture. In certain embodiments, the three-dimensional substrate culture is agitated on a stir plate in an incubator or is agitated in a bioreactor at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher). In other embodiments, a bioreactor culture is performed on a bench top, where an external motor controls an impeller inside the vessel to induce mixing. In such embodiments, gas regulation is managed using a controller and the vessel is warmed using a heat jacket. In some embodiments, the agitation occurs intermittently. In some embodiments, the agitation comprises a cycle. In some embodiments, the agitation occurs for a first time period at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by a second time period without agitation. In some instances, this cycle is repeated about 5, 10, 15, 20, 25, 30, 35, 40, 45, 50, 55, 60, 65, 70, or more times. In some instances, the first time period is about 1, 2, 3, 4, 5, 6, 7, 8, 9, 10 minutes, or more. In some instances, the first time period is about 5 minutes, or more. In some cases, the second time period is about 10 minutes, 15 minutes, 20 minutes, 25 minutes, 30 minutes, 35 minutes, 40 minutes, 45 minutes, 50 minutes, or more. In some cases, the second time period is about 30 minutes or more. In some cases, the cycle is repeated from about 32 to about 64 times (optionally about 32, 35, 40, 45, 50, 55, 60, or 64 times). In some embodiments, there are repeated cycles of agitation and no agitation in a time ratio of about 1:10 (e.g., 1 minute agitation and 10 minutes no agitation), 1:8, 1:6, 1:5, 1:4, 1:3, or 1:2. For example, the ratio of time of agitation to no agitation can be between 1:10-1:2, 1:8-1:3, or 1:7-1:4. These cycles can last at least 12 hours, at least 24 hours, at least 36 hours, at least 48 hours, or at least 72 hours. The cycle can be repeated at least 12 times, at least 24 times, at least 36 times, or at least 64 times. In some embodiment, the cycle will be repeated 24 to 64 times or 36 to 64 times. In some instances, the agitation occurs for about 5 minutes at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by about 30 minutes without agitation. This cycle may be repeated about 32-64 times (optionally about 32, 35, 40, 45, 50, 55, 60, or 64 times). In certain embodiments of the method, after about 18-36 hours (optionally 18 hours, 24 hours, 28 hours, 30 hours, or 36 hours) of intermittent agitation, the culture is then agitated continuously at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) for the remainder of the culture. Seeding may be performed at a volume that is about a quarter, a third, or a half of the final culture volume. After seeding, culture media may be added to reach full culture volume.

In some embodiments, the culture may be monitored by feeding the culture, performing at least one LIVE/DEAD™ assay on the culture, and/or assessing cell coverage on the three-dimensional substrates. The culture may be fed at intervals of about two days to about four days, with a metabolic sample taken daily and after a feed. In some embodiments, the culture may be fed more frequently or even continuously, and metabolic samples can be taken, hourly, daily, or every about 12 hours. In some instances, monitoring and/or sample measurement occurs daily during the duration of the culturing process. In other instances, monitoring and/or sample measurement occurs once daily, twice daily, or as needed to ensure correct readings of measurements. In additional instances, monitoring and/or sample measurement occurs every other day, every two days, every three days, or every four days. In further instances, monitoring and/or sample measurement occurs continuously during the duration of the culturing process. In some embodiments of the method, a sample is monitored and maintained to determine at least one of pH, glucose, lactate, glutamine, ammonium, or dissolved oxygen levels, or biocapacitance. In some embodiments of the method, a cell count is performed daily to access growth. In some instances, where cells are cultured in a bioreactor, measurements are taken through the use of a probe.

Harvesting the culture also may include at least one of and any combination of the following steps: harvesting a plurality of alveolar epithelial cells from the three-dimensional substrates by allowing the three-dimensional substrates to settle; removing a quantity of media from the cell culture vessel; washing the cell culture vessel; adding a quantity of an agent to detach the cells from or dissolve the three-dimensional substrates; agitating the cell culture vessel; collecting a cell solution into a sterile bioprocess container; rinsing the cell culture vessel; collecting a quantity of rinse from the cell culture vessel; neutralizing the detachment enzyme; spinning the quantity of cell solution; aspirating the supernatant of the pelleted cell solution; and resuspending any sample in phosphate buffered saline, cell culture medium, or cryopreservation medium. In some embodiments, harvesting is performed between about 10 to about 18 days of culture.

In some embodiments, the harvested cells are further processed. In some instances, the harvested cells are seeded onto new three-dimensional substrates and continued in culture. The agent that detaches the cells from or dissolves the three-dimensional substrate may be at least one detachment enzyme, optionally trypsin or tryp-LE. In other instances, the harvested cells are resuspended in a cryopreservation medium and subsequently frozen for storage.

In some embodiments of the methods, the plurality of harvested alveolar epithelial cells express pro-surfactant protein C (pSP-C), indicating the alveolar cells are still functional after expansion. In some preferred embodiments, the plurality of harvested alveolar epithelial cells lose no more than 30% of pSP-C expression in the first 14, 15, 18, 20, 25, 30, 35, or 40 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 14, 15, 18, 20, 25, 30, 35, or 40 days of culture. In some embodiments, the plurality of harvested alveolar epithelial cells comprise a population with pSP-C expression greater than about 30% after about 14 15, 18, 20, 25, 30, 35, or 40 days. In some instances, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 14, 15, 18, 20, 25, 30, 35, or 40 days. The plurality of harvested alveolar epithelial cells also may express the alveolar type 2 cell marker HT2-280. In certain embodiments, the plurality of harvested alveolar epithelial cells does not express an excess of CK5 or comprise an overgrowth of airway basal cells.

Microcarriers used in some embodiments may comprise a stiffness between about 1 kPa to about 100 kPa. In certain embodiments, the microcarriers comprise a stiffness of about 4 kPa.

A cell culture medium composition for culturing alveolar epithelial cells is also provided herein. In some embodiments, the composition may include a transforming growth factor-β (TGF-β) pathway inhibitor, a Wnt pathway activator, a Rho kinase (ROCK) inhibitor, an epidermal growth factor (EGF), a keratinocyte growth factor (KGF), and/or a fetal bovine serum (FBS).

In some embodiments of the composition, the TGF-β pathway inhibitor may be present in the media at a level of about 1 μM to about 10 μM. In some embodiments, the Wnt pathway activator may be present in the media at a level of about 1 μM to about 10 μM. In some embodiments, the ROCK inhibitor may be present in the media at a level of about 1 μM to about 10 μM. In some embodiments, the EGF comprises between about 25 ng/mL to about 200 ng/mL of the composition. In some embodiments, the KGF comprises between about 25 ng/mL to about 200 ng/mL of the composition. In some embodiments, the fetal bovine serum comprises about 1% to about 10% volume concentration. In certain embodiments, the TGF-β inhibitor comprises at least one of A-83-01 or DMH1. In certain embodiments, the Wnt pathway activator comprises CHIR99021. In other embodiments, the ROCK inhibitor comprises Y27632. In some cases, the cell culture medium is O-WREKT media.

In further embodiments of the composition, the composition further may include a plurality of three-dimensional substrates. In certain preferred embodiments, the three-dimensional substrates may be microcarriers.

In certain embodiments, further disclosed herein is a kit comprising a plurality of alveolar epithelial cells obtained by a method described herein or with the cell culture media composition described herein.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1 depicts images of the results of a live staining assay of AT2 cells on three-dimensional substrates over three passages.

FIG. 2 is a graphical depiction of the phenotypic stability of alveolar epithelial cell cultures on three-dimensional substrates over three passages.

FIG. 3 shows a graphical comparison across trials of the maintenance of functional alveolar type 2 cells in three-dimensional culture versus two-dimensional culture.

FIG. 4A illustrates a summary of all spinner flask AT2 microcarrier expansions performed, broken down by passage.

FIG. 4B-FIG. 4C illustrate AT2 fold change (FIG. 4B) and in-process cell counts (FIG. 4C) of the 250 mL spinner flask passage 0 expansion runs performed with GE Cultispher GL microcarriers in the O-WREKT media.

FIG. 4D illustrates AT2 cell expansion at different passages in a spinner flask via in-process cell counts and cell expansion metrics, including AT2 fold change, population doubling level, and population doubling time (hours).

FIG. 5 illustrates an exemplary bioreactor expansion process.

FIG. 6A illustrates cell growth at different scales.

FIG. 6B illustrates cell growth at different passages in a bioreactor.

FIG. 7 illustrates bioreactor expansion growth metrics from passage 0 to passage 2.

FIG. 8 illustrates bioreactor expansion phenotypic analysis.

FIG. 9 illustrates exemplary process scale up/out for use with one or more of the methods described herein.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. Although not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety. In case of conflict, the present specification, including definitions, will control. Other aspects are set forth within the claims that follow.

The practice of the present technology will employ, unless otherwise indicated, conventional techniques of tissue culture, immunology, molecular biology, microbiology, chemical engineering, and cell biology, which are within the skill of the art.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations that can be varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”.

### Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, “optional” or “optionally” means that the subsequently described event or circumstance can or cannot occur, and that the description includes instances where the event or circumstance occurs and instances where it does not.

As used herein, “microcarrier” is a support matrix allowing for the growth of cells in bioreactors. Microcarrier beads, containers, or vessels can be composed of any material suitable for tissue culture, including, but not limited to, glass, polystyrene, poly(carprolactone), nylon, poly(ethylene terephthalate) (PET), poly(clycolic acid) (PGA), gelatin, and/or dextran. Microcarriers also may comprise a material that is magnetic or can become magnetic, such as Fe₃O₄. Microcarriers can be of any suitable size and/or shape for culturing cells, with diameters typically in the range of about 25 μm to about 500 μm, but can be larger or smaller. Microcarriers can be porous (e.g., microporous or macroporous) or solid.

As used herein, the term “complete media” and “complete medium” refers to a cell culture media that are optimized for alveolar epithelial cell growth (e.g., alveolar type II epithelial cells, optionally human AT2 cells). In some instances, a complete media comprises inorganic salts, trace elements, vitamins, amino acids, lipids, carbohydrates, cytokines, growth factors, small molecules, and/or additional proteins, in which the ratio of each components has been optimized for cell growth. Exemplary additional proteins include albumin, transferrin, fibronectin, and insulin. Exemplary carbohydrates include glucose. Exemplary inorganic salts include sodium, potassium, and calcium ions. Exemplary trace elements include zinc, copper, selenium, and tricarboxylic acid. Exemplary amino acids include essential amino acids such as L-glutamine (e.g., alanyl-1-glutamine or glycyl-1-glutamine); or non-essential amino acids (NEAA) such as glycine, L-alanine, L-asparagine, L-aspartic acid, L-glutamic acid, L-proline, and/or L-serine. In some embodiments, the complete media also comprises one or more of sodium bicarbonate (NaHCO₃), HEPES (4-(2-hydroxyethyl)-1-piperazine ethanesulfonic acid), phenol red, antibiotics, and/or β-mercaptoethanol. In some instances, the complete media is a serum-free media. In some instances, the complete media is a xeno-free media.

As used herein, the term “chemically-defined media” refers to a cell culture media in which the compositions and concentrations of all components are known. It differs from a complete media in that the complete media may contain components, e.g., animal-derived components, in which the composition and/or concentration are not known. Sometimes, a complete media can also be a chemically-defined media if the compositions and concentrations of all components are known.

In some instances, a “xeno-free” media does not contain any animal-derived (non-human) component. In some instances, a xeno-free media contains one or more human-derived components such as human serum, growth factors, and insulin.

In some embodiments, a “serum-free” media does not contain serum or plasma but may contain components derived from serum or plasma. In some instances, the “serum-free” media contains animal-derived components such as bovine serum albumin (BSA).

In some embodiment, a “minimum” media comprises the minimal necessities for growth of a target cell. In some instances, the minimum media contains inorganic salts, carbon source, and water. In some instances, supplements, cytokines, and/or proteins such as albumin (e.g., HSA) are added to the minimum media. As used herein, supplements comprise trace elements, vitamins, amino acids, lipids, carbohydrates, cytokines, growth factors, or a combination thereof.

### Methods for Expansion of Alveolar Epithelial Cells

Disclosed herein, in certain embodiments, is a method for producing alveolar epithelial cells, optionally alveolar type II epithelial cells. In some aspects, the method can include: preparing a plurality of three-dimensional substrates in a cell culture vessel; seeding a plurality of alveolar epithelial cells by combining the three-dimensional substrate and the alveolar epithelial cells in the cell culture vessel and providing conditions suitable to enable attachment of the cells to the three-dimensional substrate to create a suspension culture; promoting growth of the alveolar epithelial cells on or within the three-dimensional substrates; monitoring the culture for cell proliferation; and harvesting a plurality of alveolar epithelial cells from the three-dimensional substrates. In some embodiments, the method optionally comprises seeding new three-dimensional substrates after the harvesting step to continue to grow and expand the alveolar epithelial cells. The alveolar epithelial cells may be alveolar type II epithelial cells, including human AT2 cells.

In some aspects, the three-dimensional substrates may be at least one of a solid, microporous, or macroporous three-dimensional substrates. The alveolar epithelial cells can be cultured on top of, within, or both on top of and within the three-dimensional substrates. In certain preferred embodiments, the three-dimensional substrates may be a plurality of microcarriers.

In some embodiments, the substrate is a microporous substrate. In such instances, the alveolar epithelial cells (e.g., alveolar type II epithelial cells) are cultured on top of or on the surface of the three-dimensional microporous substrates. In some cases, the microporous substrate is used in a stirred tank partial media exchange culture. In some embodiments, the microporous substrate is used in a stirred tank perfusion culture. In some embodiments, the microporous substrate may be high density microporous substrates for use in a fluidized bed perfusion culture. In other embodiments, the microporous substrates may be high density macroporous substrates for use in a packed bed perfusion culture.

In some embodiments, the substrate is a macroporous substrate. In such instances, the alveolar epithelial cells (e.g., alveolar type II epithelial cells) are cultured on top of, within, or both on top of and within the three-dimensional macroporous substrates. In some embodiments, the macroporous substrate is used in a stirred tank partial media exchange culture. In some embodiments, the macroporous substrate is used in a stirred tank perfusion culture. In some embodiments, the macroporous substrate may be high density macroporous substrate for use in a fluidized bed perfusion culture. In other embodiments, the macroporous substrate may be high density macroporous substrate for use in a packed bed perfusion culture.

In some embodiments, the substrate is a solid substrate. In such instances, the alveolar epithelial cells (e.g., alveolar type II epithelial cells) are cultured on top of or on the surface of the three-dimensional solid substrates. In some cases, the solid substrate is used in a stirred tank partial media exchange culture. In some embodiments, the solid substrate is used in a stirred tank perfusion culture. In some embodiments, the solid substrate may be high density solid substrate for use in a fluidized bed perfusion culture. In other embodiments, the solid substrate may be high density solid substrate for use in a packed bed perfusion culture.

The cell culture vessel may be a spinner flask or bioreactor and the three-dimensional substrates comprise about 1-10 mg/mL in the culture. In some instances, the three-dimensional substrates comprises about 1 mg/mL, 1.2 mg/mL, 1.4 mg/mL, 1.5 mg/mL, 1.6 mg/mL, 1.8 mg/mL, 2 mg/mL, 3 mg/mL, 4 mg/mL, 5 mg/mL, 6 mg/mL, 7 mg/mL, 8 mg/mL, 9 mg/mL, or 10 mg/mL in the culture. In some cases, the three-dimensional substrates comprises about 1 mg/mL in the culture. In some cases, the three-dimensional substrates comprises about 1.2 mg/mL in the culture. In some cases, the three-dimensional substrates comprises about 1.4 mg/mL in the culture. In some cases, the three-dimensional substrates comprises about 1.5 mg/mL in the culture. In some cases, the three-dimensional substrates comprises about 1.6 mg/mL in the culture. In some cases, the three-dimensional substrates comprises about 1.8 mg/mL in the culture. In some cases, the three-dimensional substrates comprises about 2 mg/mL in the culture.

In some embodiments, the cell culture vessel is a spinner flask, and the culture may be at least a 125 mL, 250 mL, 500 mL, 1 L, 3 L, or 10 L culture. In some embodiments, the culture may be at least a 125 mL, 250 mL, or 1 L culture.

In some embodiments, the cell culture vessel is a bioreactor and the culture may be at least a 1 L, 3 L, 3.75 L, 5 L, 7 L, 10 L, 15 L, 20 L, 25 L, 30 L, 35 L, 40 L, or 60 L culture. In some cases, the culture may be at least a 3 L, 3.75 L, 10 L, or 40 L culture.

Cell seeding further may include adding alveolar epithelial cells (e.g., either freshly isolated or cryopreserved) in cell culture media to the cell culture vessel. Seeding may also include agitating the culture. For example, in some embodiments of the disclosed methods, the three-dimensional substrate culture may be agitated on a stir plate in an incubator at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher). In certain embodiments of the method, the agitation occurs intermittently, and comprises a cycle where agitation occurs for a first time period at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by a second time period without agitation. In some instances, this cycle is repeated about 5, 10, 15, 20, 25, 30, 35, 40, 45, 50, 55, 60, 65, 70, or more times. In some instances, the first time period is about 1, 2, 3, 4, 5, 6, 7, 8, 9, 10 minutes, or more. In some instances, the first time period is about 5 minutes, or more. In some cases, the second time period is about 10 minutes, 15 minutes, 20 minutes, 25 minutes, 30 minutes, 35 minutes, 40 minutes, 45 minutes, 50 minutes, or more. In some cases, the second time period is about 30 minutes or more. In some cases, the cycle is repeated from about 32 to about 64 times (optionally about 32, 35, 40, 45, 50, 55, 60, or 64 times). In some embodiments, there are repeated cycles of agitation and no agitation in a time ratio of about 1:10 (e.g., 1 minute agitation and 10 minutes no agitation), 1:8, 1:6, 1:5, 1:4, 1:3, or 1:2. For example, the ratio of time of agitation to no agitation can be between 1:10-1:2, 1:8-1:3, or 1:7-1:4. These cycles can last at least 12 hours, at least 24 hours, at least 36 hours, at least 48 hours, or at least 72 hours. The cycle can be repeated at least 12 times, at least 24 times, at least 36 times, or at least 64 times. In some embodiment, the cycle will be repeated 24 to 64 times or 36 to 64 times. In some instances, the agitation occurs intermittently, and includes a cycle where agitation occurs for about 5 minutes at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by about 30 minutes without agitation, after which this cycle is repeated about 32-64 times (optionally about 32, 35, 40, 45, 50, 55, 60, or 64 times). In some embodiments, after about 18-36 hours (optionally 18 hours, 24 hours, 28 hours, 30 hours, or 36 hours) the culture may be agitated at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) continuously until harvesting the cells. In some instances, seeding is performed at a volume that is about a quarter, a third, or a half of the final culture volume. After seeding, culture media may be added to reach full culture volume.

In some embodiments, the three-dimensional substrate culture may be agitated in a 250 mL spinner flask on a stir plate in an incubator at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher). In such instances, the three-dimensional substrate culture may be agitated on the stir plate in an incubator at about 25 RPM or higher, 30 RPM or higher, 35 RPM or higher, or 40 RPM or higher. In some cases, the three-dimensional substrate culture may be agitated on the stir plate in an incubator at about 35 RPM or higher. In some embodiments, the agitation occurs intermittently, and comprises a cycle where agitation occurs for a first time period at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by a second time period without agitation. In some instances, this cycle is repeated about 5, 10, 15, 20, 25, 30, 35, 40, 45, 50, 55, 60, 65, 70, or more times. In some instances, the first time period is about 1, 2, 3, 4, 5, 6, 7, 8, 9, 10 minutes, or more. In some instances, the first time period is about 5 minutes, or more. In some cases, the second time period is about 10 minutes, 15 minutes, 20 minutes, 25 minutes, 30 minutes, 35 minutes, 40 minutes, 45 minutes, 50 minutes, or more. In some cases, the second time period is about 30 minutes or more. In some cases, the cycle is repeated from about 32 to about 64 times (optionally about 32, 35, 40, 45, 50, 55, 60, or 64 times). In some embodiments, there are repeated cycles of agitation and no agitation in a time ratio of about 1:10 (e.g., 1 minute agitation and 10 minutes no agitation), 1:8, 1:6, 1:5, 1:4, 1:3, or 1:2. For example, the ratio of time of agitation to no agitation can be between 1:10-1:2, 1:8-1:3, or 1:7-1:4. These cycles can last at least 12 hours, at least 24 hours, at least 36 hours, at least 48 hours, or at least 72 hours. The cycle can be repeated at least 12 times, at least 24 times, at least 36 times, or at least 64 times. In some embodiment, the cycle will be repeated 24 to 64 times or 36 to 64 times. In some cases, the agitation occurs intermittently, optionally comprising a cycle where agitation occurs for about 5 minutes at about 35 RPM or higher followed by about 30 minutes without agitation, after which this cycle is repeated about 32-64 times. In some embodiments, after about 18-36 hours (optionally 18 hours, 24 hours, 28 hours, 30 hours, or 36 hours) the culture may be agitated at about 35 RPM or higher continuously until harvesting the cells. In some instances, seeding is performed at a volume that is about a quarter, a third, or a half of the final culture volume. After seeding, culture media may be added to reach full culture volume.

In some embodiments, the three-dimensional substrate culture may be agitated in a 1 L spinner flask on a stir plate in an incubator at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher). In such instances, the three-dimensional substrate culture may be agitated on the stir plate in an incubator at about 20 RPM or higher, 25 RPM or higher, 30 RPM or higher, or 35 RPM or higher. In some cases, the three-dimensional substrate culture may be agitated on the stir plate in an incubator at about 20 RPM or higher. In some embodiments, the agitation occurs intermittently, and comprises a cycle where agitation occurs for a first time period at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by a second time period without agitation. In some instances, this cycle is repeated about 5, 10, 15, 20, 25, 30, 35, 40, 45, 50, 55, 60, 65, 70, or more times. In some instances, the first time period is about 1, 2, 3, 4, 5, 6, 7, 8, 9, 10 minutes, or more. In some instances, the first time period is about 5 minutes, or more. In some cases, the second time period is about 10 minutes, 15 minutes, 20 minutes, 25 minutes, 30 minutes, 35 minutes, 40 minutes, 45 minutes, 50 minutes, or more. In some cases, the second time period is about 30 minutes or more. In some cases, the cycle is repeated from about 32 to about 64 times (optionally about 32, 35, 40, 45, 50, 55, 60, or 64 times). In some embodiments, there are repeated cycles of agitation and no agitation in a time ratio of about 1:10 (e.g., 1 minute agitation and 10 minutes no agitation), 1:8, 1:6, 1:5, 1:4, 1:3, or 1:2. For example, the ratio of time of agitation to no agitation can be between 1:10-1:2, 1:8-1:3, or 1:7-1:4. These cycles can last at least 12 hours, at least 24 hours, at least 36 hours, at least 48 hours, or at least 72 hours. The cycle can be repeated at least 12 times, at least 24 times, at least 36 times, or at least 64 times. In some embodiment, the cycle will be repeated 24 to 64 times or 36 to 64 times. In some cases, the agitation occurs intermittently, optionally comprising a cycle where agitation occurs for about 5 minutes at about 20 RPM or higher followed by about 30 minutes without agitation, after which this cycle is repeated about 32-64 times. In some embodiments, after about 18-36 hours (optionally 18 hours, 24 hours, 28 hours, 30 hours, or 36 hours) the culture may be agitated at about 20 RPM or higher continuously until harvesting the cells. In some instances, seeding is performed at a volume that is about a quarter, a third, or a half of the final culture volume. After seeding, culture media may be added to reach full culture volume.

In some embodiments, the three-dimensional substrate culture may be agitated in a bioreactor at about 20 RPM or higher (optionally 25 RPM or higher, 28 RPM or higher, 30 RPM or higher, 32 RPM or higher, 35 RPM or higher, 38 RPM or higher, 39 RPM or higher, 40 RPM or higher, 42 RPM or higher, 45 RPM or higher, 48 RPM or higher, 50 RPM or higher, 52 RPM or higher, 55 RPM or higher, or 60 RPM or higher). In some instances, the bioreactor culture is performed on a bench top, where an external motor controls an impeller inside the vessel to induce mixing. In such embodiments, gas regulation is managed using a controller and the vessel is warmed using a heat jacket. In certain embodiments of the method, the agitation may occur intermittently, and may comprise a cycle where agitation occurs for a first time period at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by a second time period without agitation. In some instances, this cycle is repeated about 5, 10, 15, 20, 25, 30, 35, 40, 45, 50, 55, 60, 65, 70, or more times. In some instances, the first time period is about 1, 2, 3, 4, 5, 6, 7, 8, 9, 10 minutes, or more. In some instances, the first time period is about 5 minutes, or more. In some cases, the second time period is about 10 minutes, 15 minutes, 20 minutes, 25 minutes, 30 minutes, 35 minutes, 40 minutes, 45 minutes, 50 minutes, or more. In some cases, the second time period is about 30 minutes or more. In some cases, the cycle is repeated from about 32 to about 64 times (optionally about 32, 35, 40, 45, 50, 55, 60, or 64 times). In some embodiments, there are repeated cycles of agitation and no agitation in a time ratio of about 1:10 (e.g., 1 minute agitation and 10 minutes no agitation), 1:8, 1:6, 1:5, 1:4, 1:3, or 1:2. For example, the ratio of time of agitation to no agitation can be between 1:10-1:2, 1:8-1:3, or 1:7-1:4. These cycles can last at least 12 hours, at least 24 hours, at least 36 hours, at least 48 hours, or at least 72 hours. The cycle can be repeated at least 12 times, at least 24 times, at least 36 times, or at least 64 times. In some embodiment, the cycle will be repeated 24 to 64 times or 36 to 64 times. In some embodiments, the agitation occurs intermittently and includes a cycle where agitation occurs for about 5 minutes at about 20 RPM or higher (optionally 25 RPM or higher, 28 RPM or higher, 30 RPM or higher, 32 RPM or higher, 35 RPM or higher, 38 RPM or higher, 39 RPM or higher, 40 RPM or higher, 42 RPM or higher, 45 RPM or higher, 48 RPM or higher, 50 RPM or higher, 52 RPM or higher, 55 RPM or higher, or 60 RPM or higher) followed by about 30 minutes without agitation, after which this cycle is repeated about 32-64 times (optionally about 32, 35, 40, 45, 50, 55, 60, or 64 times). In some embodiments, after about 18-36 hours (optionally 18 hours, 24 hours, 28 hours, 30 hours, or 36 hours) the culture may be agitated at about 20 RPM or higher (optionally 25 RPM or higher, 28 RPM or higher, 30 RPM or higher, 32 RPM or higher, 35 RPM or higher, 38 RPM or higher, 39 RPM or higher, 40 RPM or higher, 42 RPM or higher, 45 RPM or higher, 48 RPM or higher, 50 RPM or higher, 52 RPM or higher, 55 RPM or higher, or 60 RPM or higher) continuously until harvesting the cells. After seeding, culture media may be added to reach full culture volume.

In some embodiments, the three-dimensional substrate culture may be agitated in a 1 L bioreactor at about 20 RPM or higher (optionally 25 RPM or higher, 28 RPM or higher, 30 RPM or higher, 32 RPM or higher, 35 RPM or higher, 38 RPM or higher, 39 RPM or higher, 40 RPM or higher, 42 RPM or higher, 45 RPM or higher, 48 RPM or higher, 50 RPM or higher, 52 RPM or higher, 55 RPM or higher, or 60 RPM or higher). In such instances, the three-dimensional substrate culture may be agitated in the 1 L bioreactor at about 45 RPM or higher, 48 RPM or higher, 50 RPM or higher, or 52 RPM or higher. In some cases, the three-dimensional substrate culture may be agitated in the 1 L bioreactor at about 48 RPM or higher or 52 RPM or higher. In some embodiments, the agitation occurs intermittently, and comprises a cycle where agitation occurs for a first time period at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by a second time period without agitation. In some instances, this cycle is repeated about 5, 10, 15, 20, 25, 30, 35, 40, 45, 50, 55, 60, 65, 70, or more times. In some instances, the first time period is about 1, 2, 3, 4, 5, 6, 7, 8, 9, 10 minutes, or more. In some instances, the first time period is about 5 minutes, or more. In some cases, the second time period is about 10 minutes, 15 minutes, 20 minutes, 25 minutes, 30 minutes, 35 minutes, 40 minutes, 45 minutes, 50 minutes, or more. In some cases, the second time period is about 30 minutes or more. In some cases, the cycle is repeated from about 32 to about 64 times (optionally about 32, 35, 40, 45, 50, 55, 60, or 64 times). In some embodiments, there are repeated cycles of agitation and no agitation in a time ratio of about 1:10 (e.g., 1 minute agitation and 10 minutes no agitation), 1:8, 1:6, 1:5, 1:4, 1:3, or 1:2. For example, the ratio of time of agitation to no agitation can be between 1:10-1:2, 1:8-1:3, or 1:7-1:4. These cycles can last at least 12 hours, at least 24 hours, at least 36 hours, at least 48 hours, or at least 72 hours. The cycle can be repeated at least 12 times, at least 24 times, at least 36 times, or at least 64 times. In some embodiment, the cycle will be repeated 24 to 64 times or 36 to 64 times. In some cases, the agitation occurs intermittently, optionally comprising a cycle where agitation occurs for about 5 minutes at about 48 RPM or higher followed by about 30 minutes without agitation, after which this cycle is repeated about 32-64 times. In some embodiments, after about 18-36 hours (optionally 18 hours, 24 hours, 28 hours, 30 hours, or 36 hours) the culture may be agitated at about 48 RPM or higher continuously until harvesting the cells. In some instances, seeding is performed at a volume that is about a quarter, a third, or a half of the final culture volume. After seeding, culture media may be added to reach full culture volume.

In some embodiments, the three-dimensional substrate culture may be agitated in a 3.75 L bioreactor at about 20 RPM or higher (optionally 25 RPM or higher, 28 RPM or higher, 30 RPM or higher, 32 RPM or higher, 35 RPM or higher, 38 RPM or higher, 39 RPM or higher, 40 RPM or higher, 42 RPM or higher, 45 RPM or higher, 48 RPM or higher, 50 RPM or higher, 52 RPM or higher, 55 RPM or higher, or 60 RPM or higher). In such instances, the three-dimensional substrate culture may be agitated in the 3.75 L bioreactor at about 45 RPM or higher, 50 RPM or higher, 55 RPM or higher, or 60 RPM or higher. In some cases, the three-dimensional substrate culture may be agitated in the 3.75 L bioreactor at about 50 RPM or higher or 55 RPM or higher. In some embodiments, the agitation occurs intermittently, and comprises a cycle where agitation occurs for a first time period at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by a second time period without agitation. In some instances, this cycle is repeated about 5, 10, 15, 20, 25, 30, 35, 40, 45, 50, 55, 60, 65, 70, or more times. In some instances, the first time period is about 1, 2, 3, 4, 5, 6, 7, 8, 9, 10 minutes, or more. In some instances, the first time period is about 5 minutes, or more. In some cases, the second time period is about 10 minutes, 15 minutes, 20 minutes, 25 minutes, 30 minutes, 35 minutes, 40 minutes, 45 minutes, 50 minutes, or more. In some cases, the second time period is about 30 minutes or more. In some cases, the cycle is repeated from about 32 to about 64 times (optionally about 32, 35, 40, 45, 50, 55, 60, or 64 times). In some embodiments, there are repeated cycles of agitation and no agitation in a time ratio of about 1:10 (e.g., 1 minute agitation and 10 minutes no agitation), 1:8, 1:6, 1:5, 1:4, 1:3, or 1:2. For example, the ratio of time of agitation to no agitation can be between 1:10-1:2, 1:8-1:3, or 1:7-1:4. These cycles can last at least 12 hours, at least 24 hours, at least 36 hours, at least 48 hours, or at least 72 hours. The cycle can be repeated at least 12 times, at least 24 times, at least 36 times, or at least 64 times. In some embodiment, the cycle will be repeated 24 to 64 times or 36 to 64 times. In some cases, the agitation occurs intermittently, optionally comprising a cycle where agitation occurs for about 5 minutes at about 50 RPM or higher followed by about 30 minutes without agitation, after which this cycle is repeated about 32-64 times. In some embodiments, after about 18-36 hours (optionally 18 hours, 24 hours, 28 hours, 30 hours, or 36 hours) the culture may be agitated at about 50 RPM or higher continuously until harvesting the cells. In some instances, seeding is performed at a volume that is about a quarter, a third, or a half of the final culture volume. After seeding, culture media may be added to reach full culture volume.

In some embodiments, the three-dimensional substrate culture may be agitated in a 10 L bioreactor at about 20 RPM or higher (optionally 25 RPM or higher, 28 RPM or higher, 30 RPM or higher, 32 RPM or higher, 35 RPM or higher, 38 RPM or higher, 39 RPM or higher, 40 RPM or higher, 42 RPM or higher, 45 RPM or higher, 48 RPM or higher, 50 RPM or higher, 52 RPM or higher, 55 RPM or higher, or 60 RPM or higher). In such instances, the three-dimensional substrate culture may be agitated in the 10 L bioreactor at about 35 RPM or higher, 39 RPM or higher, 40 RPM or higher, or 42 RPM or higher. In some cases, the three-dimensional substrate culture may be agitated in the 10 L bioreactor at about 39 RPM or higher or 42 RPM or higher. In some embodiments, the agitation occurs intermittently, and comprises a cycle where agitation occurs for a first time period at about 20 RPM or higher (optionally 25 RPM or higher, 28 RPM or higher, 30 RPM or higher, 32 RPM or higher, 35 RPM or higher, 38 RPM or higher, 40 RPM or higher, 50 RPM or higher, or 60 RPM or higher) followed by a second time period without agitation. In some instances, this cycle is repeated about 5, 10, 15, 20, 25, 30, 35, 40, 45, 50, 55, 60, 65, 70, or more times. In some instances, the first time period is about 1, 2, 3, 4, 5, 6, 7, 8, 9, 10 minutes, or more. In some instances, the first time period is about 5 minutes, or more. In some cases, the second time period is about 10 minutes, 15 minutes, 20 minutes, 25 minutes, 30 minutes, 35 minutes, 40 minutes, 45 minutes, 50 minutes, or more. In some cases, the second time period is about 30 minutes or more. In some cases, the cycle is repeated from about 32 to about 64 times (optionally about 32, 35, 40, 45, 50, 55, 60, or 64 times). In some embodiments, there are repeated cycles of agitation and no agitation in a time ratio of about 1:10 (e.g., 1 minute agitation and 10 minutes no agitation), 1:8, 1:6, 1:5, 1:4, 1:3, or 1:2. For example, the ratio of time of agitation to no agitation can be between 1:10-1:2, 1:8-1:3, or 1:7-1:4. These cycles can last at least 12 hours, at least 24 hours, at least 36 hours, at least 48 hours, or at least 72 hours. The cycle can be repeated at least 12 times, at least 24 times, at least 36 times, or at least 64 times. In some embodiment, the cycle will be repeated 24 to 64 times or 36 to 64 times. In some cases, the agitation occurs intermittently, optionally comprising a cycle where agitation occurs for about 5 minutes at about 39 RPM or higher followed by about 30 minutes without agitation, after which this cycle is repeated about 32-64 times. In some embodiments, after about 18-36 hours (optionally 18 hours, 24 hours, 28 hours, 30 hours, or 36 hours) the culture may be agitated at about 39 RPM or higher continuously until harvesting the cells. In some instances, seeding is performed at a volume that is about a quarter, a third, or a half of the final culture volume. After seeding, culture media may be added to reach full culture volume.

In some embodiments, the culture is monitored for growth and expansion throughout the duration of the culturing process. In some embodiments, monitoring may include any of feeding the culture, performing at least one LIVE/DEAD™ assay on the culture, and assessing cell coverage on the three-dimensional substrates. The culture may be fed at intervals of about two days to about four days, with a metabolic sample taken, e.g., daily, and/or after a feed. In some instances, monitoring and/or sample measurement occurs daily during the duration of the culturing process. In other instances, monitoring and/or sample measurement occurs once daily, twice daily, or as needed to ensure correct readings of measurements. In additional instances, monitoring and/or sample measurement occurs every other day, every two days, every three days, or every four days. In further instances, monitoring and/or sample measurement occurs continuously during the duration of the culturing process. Through monitoring, at least one of and any combination of pH, glucose, lactate, glutamine, ammonium, and/or dissolved oxygen levels and/or biocapacitance may be monitored. In some cases, exemplary low and high levels of pH, glucose, lactate, glutamine, ammonium, and/or dissolved oxygen are illustrated in Table 1. In some embodiments of the method, cell count is performed daily to access growth. In some instances, where cells are cultured in a bioreactor, measurements are taken through use of a probe.

TABLE 1

| Metabolite | Set Point | Low | | High | |
|---|---|---|---|---|---|
| pH (Spinner flask) | N/A | 7.0 | | 7.8 | |
| pH (Bioreactor) | 7.3 +/− 0.1 | 7.3 | | 7.4 | |
| Dissolved Oxygen* | 10% | 1% | | 22% | |
| Glucose | N/A | 1.5 | g/L | 3.2 | g/L |
| Glutamine | N/A | 0 | mmol | 2 | mmol |
| Lactate | N/A | 0 | g/L | 1.5 | g/L |
| Ammonium | N/A | 0 | mmol | 1.5 | mmol |

*Dissolved oxygen percentage is based on 100% oxygen, rather than 100% air (which comprises about 21% oxygen).

In some embodiments of the disclosed methods, harvesting a plurality of alveolar epithelial cells from the three-dimensional substrates may include the following: allowing the three-dimensional substrates to settle and removing a quantity of media from the cell culture vessel; washing the cell culture vessel; adding a quantity of an agent to detach the cells from the three-dimensional substrates; agitating the cell culture vessel; collecting the cell solution into a sterile bioprocess container; rinsing the cell culture vessel; collecting a quantity of rinse from the cell culture vessel; neutralizing the detachment enzyme; spinning the quantity of cell solution; aspirating the supernatant of the pelleted cell solution; and resuspending any sample in phosphate buffered saline, cell culture medium, or cryopreservation medium. In some embodiments, harvesting is performed from about 10 to about 18 days of culture, from about 12-16 days of culture, or from about 12-14 days of culture. After harvesting, the harvested cells may be seeded onto new three-dimensional substrates and continued in culture.

The cells can also be detached from the three-dimensional substrates or the three-dimensional substrates can be dissolved. In some embodiments of the disclosed methods, the agent to detach the cells from or dissolve the three-dimensional substrate may be at least one detachment enzyme, optionally trypsin or tryp-LE.

The plurality of harvested alveolar epithelial cells may also express certain biological markers as a measure of cell health, adhesion, or other indicators denoting successful expansion. The plurality of harvested alveolar epithelial cells further may express HT2-280, a biomarker specific to apical plasma, which has the biochemical characteristics of an integral membrane protein. HT2-280 is an identity marker of AT2 cells, which demonstrates that there is a plurality of AT2 cells in the culture. In some embodiments of the method, the plurality of harvested epithelial cells may express pSP-C. In some preferred embodiments, the plurality of harvested alveolar epithelial cells lose no more than 30% of pSP-C expression in the first 14, 15, 18, 20, 25, 30, 35, or 40 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 14, 15, 18, 20, 25, 30, 35, or 40 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 14 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 15 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 18 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 20 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 25 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 30 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 35 days of culture. In some embodiments, the plurality of harvested alveolar epithelial cells lose no more than 29%, 28%, 27%, 26%, 25%, 24%, 23%, 22%, 21%, 20%, 18%, 16%, 15%, 12%, 10%, 8%, 6%, 5%, 4%, 3%, 2%, or 1% of pSP-C expression in the first 40 days of culture.

In some embodiments, the plurality of harvested alveolar epithelial cells comprise a population with pSP-C expression greater than about 30% after about 14 15, 18, 20, 25, 30, 35, or 40 days. In some instances, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 14, 15, 18, 20, 25, 30, 35, or 40 days. In some cases, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 14 days. In some cases, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 15 days. In some cases, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 18 days. In some cases, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 20 days. In some cases, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 25 days. In some cases, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 30 days. In some cases, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 35 days. In some cases, the plurality of harvested alveolar epithelial cells comprise a population with pSP-C expression greater than about 30%, 40%, 41%, 50%, 60%, 70%, 72%, 74%, 75%, 79%, 80%, 85%, 86%, 90%, 91%, 92%, 93%, 94%, 95%, 96%, 97%, 98%, or 99% after about 40 days.

pSP-C is a functional marker, which may indicate that more pSP-C expression has been retained than in two-dimensional culture. In some embodiments of the method, the plurality of harvested alveolar epithelial cells may not express an excess of CK5 or include an overgrowth of airway basal cells.

Microcarriers used in some embodiments of the disclosed methods may comprise a stiffness between about 1 kPa to about 100 kPa. In certain embodiments, the microcarriers comprise a stiffness of about 4 kPa (e.g., within 10% or 20% of 4 kPa), which is within a range configured to mimic the stiffness of the lung, especially human lung. In some instances, the microcarriers are solid, microporous, or macroporous. In some cases, about 1-10 mg/mL of the microcarriers are added to a cell culture vessel. In some instances, about 1 mg/mL, 1.2 mg/mL, 1.4 mg/mL, 1.5 mg/mL, 1.6 mg/mL, 1.8 mg/mL, 2 mg/mL, 3 mg/mL, 4 mg/mL, 5 mg/mL, 6 mg/mL, 7 mg/mL, 8 mg/mL, 9 mg/mL, or 10 mg/mL of the microcarriers are added to a cell culture vessel. In some cases, about 1 mg/mL of the microcarriers are added to a cell culture vessel. In some cases, about 1.2 mg/mL of the microcarriers are added to a cell culture vessel. In some cases, about 1.4 mg/mL of the microcarriers are added to a cell culture vessel. In some cases, about 1.5 mg/mL of the microcarriers are added to a cell culture vessel. In some cases, about 1.6 mg/mL of the microcarriers are added to a cell culture vessel. In some cases, about 1.8 mg/mL of the microcarriers are added to a cell culture vessel. In some cases, about 2 mg/mL of the microcarriers are added to a cell culture vessel.

In some embodiments, cultured alveolar epithelial cells comprises about 70%, 75%, 80%, 81%, 82%, 83%, 84%, 85%, 86%, 87%, 88%, 89%, 90%, or higher of cells in a culture. In some cases, cultured alveolar epithelial cells comprises about 70% of cells in the culture. In some cases, cultured alveolar epithelial cells comprises about 75% of cells in the culture. In some cases, cultured alveolar epithelial cells comprises about 80% of cells in the culture. In some cases, cultured alveolar epithelial cells comprises about 85% of cells in the culture. In some cases, cultured alveolar epithelial cells comprises about 90% of cells in the culture.

In some embodiments, a culture described herein comprises less than about 30%, 28%, 25%, 24%, 22%, 20%, 18%, 16%, 15%, 13%, 12%, 10%, 8%, or 5% of contaminants. In some instances, the contaminants comprises undesirable cells, e.g., cells that are not alveolar epithelial cells, optionally cells that are not alveolar type II epithelial (AT2) cells, and further optionally cells that are not human AT2 cells.

In some embodiments, alveolar epithelial cells are cultured and expanded in one or more passages, two or more passages, three or more passages, four or more passages, five or more passages, or six or more passages. In some instances, the alveolar epithelial cells are cultured and expanded in 1, 2, 3, 4, 5, 6, or more passages. In some cases, the number of cells of a passage is increased by 1-fold, 2-fold, 3-fold, 4-fold, 5-fold, 6-fold, 6.5-fold, 6.6-fold, 7-fold, 8-fold, 9-fold, 10-fold, 11-fold, 12-fold, 15-fold, 20-fold, 50-fold, 100-fold, 1000-fold, or more. In some cases, the number of cells collected from one passage is increased by 1-fold. In some cases, the number of cells collected from one passage is increased by 2-fold. In some cases, the number of cells collected from one passage is increased by 5-fold. In some cases, the number of cells collected from one passage is increased by 6-fold, 6.5-fold, or 6.6-fold. In some cases, the number of cells collected from one passage is increased by 8-fold. In some cases, the number of cells collected from passage is increased by 10-fold. In some cases, the number of cells collected from passage 0 (P0) is increased by 1-fold, 2-fold, 3-fold, 4-fold, 5-fold, 6-fold, 6.5-fold, 6.6-fold, 7-fold, 8-fold, 9-fold, 10-fold, 11-fold, 12-fold, 15-fold, 20-fold, 50-fold, 100-fold, 1000-fold, or more. In some cases, the number of cells collected from passage 1 (P1) is increased by 1-fold, 2-fold, 3-fold, 4-fold, 5-fold, 6-fold, 6.5-fold, 6.6-fold, 7-fold, 8-fold, 9-fold, 10-fold, 11-fold, 12-fold, 15-fold, 20-fold, 50-fold, 100-fold, 1000-fold, or more. In some cases, the number of cells collected from passage 2 (P2) is increased by 1-fold, 2-fold, 3-fold, 4-fold, 5-fold, 6-fold, 6.5-fold, 6.6-fold, 7-fold, 8-fold, 9-fold, 10-fold, 11-fold, 12-fold, 15-fold, 20-fold, 50-fold, 100-fold, 1000-fold, or more.

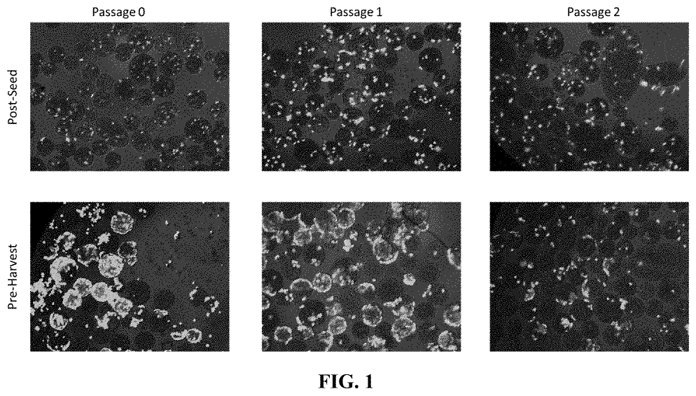

Using the methods of the disclosure, FIG. 1 depicts images of the results of a live staining assay of AT2 cells on three-dimensional substrates over three passages. Images of cells post-seed (top panel) and pre-harvest (bottom panel) for all three passages show an increase in cell coverage within microcarriers (white: live cells).

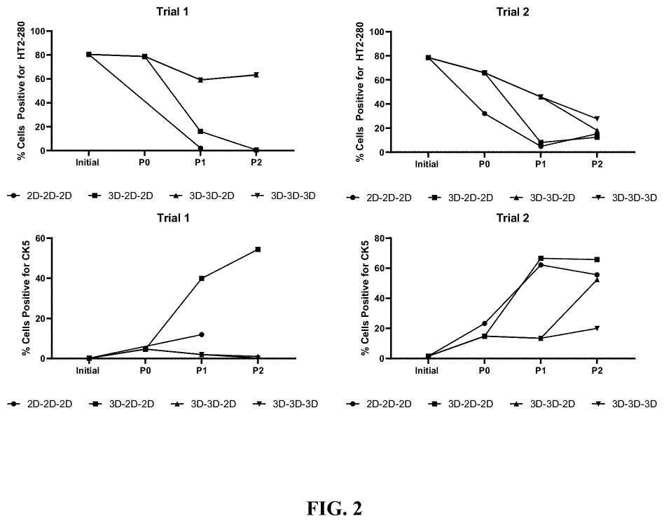

FIG. 2 illustrates a graphical depiction of the phenotypic stability of alveolar epithelial cell cultures on three-dimensional substrates over three passages. HT2-280 is an identity marker of AT2 cells. The top panel of FIG. 2 shows that HT2-280 expression was better maintained in three-dimensional culture compared to two-dimensional culture for both culture trials. Overgrowth of airway basal cells (marked by CK5 expression) is a significant challenge in AT2 expansion in standard culture. Data in the bottom panel demonstrates that CK5 expression increases more in two-dimensional culture or when cells are switched from three-dimensional to two-dimensional culture. Thus, 3D culture conditions can decrease the amount of airway basal cells in a culture medium relative to 2D culture conditions. 2D/3D designations in the chart key represent culture platform in chronological order across 3 passages, with passages separated by hyphen.

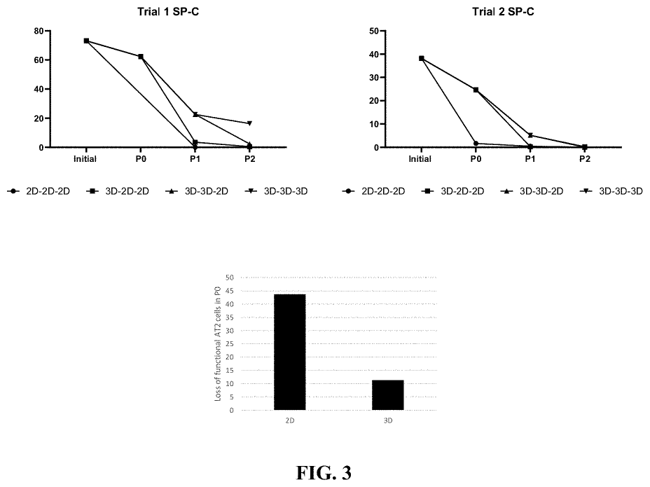

FIG. 3 shows a graphical comparison across trials of the maintenance of functional AT2 cells in three-dimensional culture versus two-dimensional culture. Pro surfactant protein C is a key functional marker of an AT2 cell, as AT2 cells primarily act to produce surfactant in the lung to reduce surface tension. The top panel of FIG. 3 depicts pSP-C expression across three passages for n=2 sample sets, demonstrating prolonged maintenance of pSP-C expression in three-dimensional cultures compared to two-dimensional. In the bottom panel of FIG. 3, the loss of functional AT2 cells in passage 0 was calculated as the difference in the percentage of pSP-C positive cells (pSP-C+) divided by percentage of HT2-280 positive (HT2-280+) cells from the initial culture to passage 0; this calculation is performed assuming all pSP-C+ cells are HT2-280+. This data demonstrates there is an about 44% loss in the percent of functional AT2 cells in two-dimensional culture compared to an about 11% loss in three-dimensional culture, indicating that AT2 cells better maintain function on three-dimensional culture substrates compared to two-dimensional. 3D culture conditions can increase the amount of pSP-C+ cells in a culture medium relative to 2D culture conditions.

The culture yield will depend on a variety of factors including the culture time, conditions, and volume. The methods described herein can result in a yield of at least 1×10⁶ cells/culture, 1×10⁷ cells/culture, 1×10⁸ cells/culture, 1×10⁹ cells/culture, or 5×10⁹ cells/culture. In some embodiments, the cell yield will be at least 1×10⁵ cells/mL, 2×10⁵ cells/mL, 3×10⁵ cells/mL, 4×10⁵ cells/mL, or 5×10⁵ cells/mL. These resulting cells can be AT2 cells or cells that have one or more functional characteristics of AT2 cells.

In some embodiments, alveolar epithelial cells are cultured and expanded using one or more methods described herein and/or with a media composition described herein for use in regenerative medicine, e.g., for use in tissue or organ engineering. In some embodiments, the cultured alveolar epithelial cells using one or more methods described herein and/or with a media composition described herein are also used for cell therapy, e.g., for the treatment of one or more diseases or conditions such as cancer.

### Compositions for Culturing Alveolar Epithelial Cells

A cell culture media composition for culturing alveolar epithelial cells is also provided herein. In some aspects, the cell culture media composition for culturing alveolar epithelial cells may include: a TGF-β pathway inhibitor; a Wnt pathway activator; a ROCK inhibitor; an epidermal growth factor (EGF); a keratinocyte growth factor (KGF); and a fetal bovine serum (FBS). In some instances, the cell culture media is a complete cell media, optionally supplemented with one or more of a TGF-β pathway inhibitor, a Wnt pathway activator, a ROCK inhibitor, an EGF, or a KGF. In some instances, the cell culture media is an FBS-based media, optionally supplemented with one or more of a TGF-β pathway inhibitor, a Wnt pathway activator, a ROCK inhibitor, an EGF, or a KGF. In some instances, the cell culture media is a serum-free media, optionally supplemented with one or more of a TGF-β pathway inhibitor, a Wnt pathway activator, a ROCK inhibitor, an EGF, or a KGF. In some instances, the cell media is a chemically-defined media, optionally supplemented with one or more of a TGF-β pathway inhibitor, a Wnt pathway activator, a ROCK inhibitor, an EGF, a KGF, or FBS. In some instances, the cell media is a minimum media, optionally supplemented with one or more of a TGF-β pathway inhibitor, a Wnt pathway activator, a ROCK inhibitor, an EGF, a KGF, or FBS. In some instances, the cell media further comprises one or more amino acid supplements (e.g., L-glutamine) and/or antibiotics. In some cases, the cell culture media composition is used with a method described supra for culturing alveolar epithelial cells, optionally alveolar type II epithelial cells, further optionally human AT2 cells.

In some embodiments of the media composition, the TGF-β pathway inhibitor may be from about 1 μM to about 10 μM in molar concentration, or any value or subrange there between. For product concentrations, the TGF-β pathway inhibitor further may be included at a molar concentration of about 1.25 μM, 1.5 μM, 1.75 μM, 2.0 μM, 2.25 μM, 2.5 μM, 2.75 μM, 3.0 μM, 3.25 μM, 3.5 μM, 3.75 μM, 4.0 μM, 4.25 μM, 4.5 μM, 4.75 μM, 5.0 μM, 5.25 μM, 5.5 μM, 5.75 μM, 6.0 μM, 6.25 μM, 6.5 μM, 6.75 μM, 7.0 μM, 7.25 μM, 7.5 μM, 7.75 μM, 8.0 μM, 8.25 μM, 8.5 μM, 8.75 μM, 9.0 μM, 9.25 μM, 9.5 μM, 9.75 μM, or 10.0 μM. In some instances, the TGF-β pathway inhibitor is included at a molar concentration of about 1 μM or about 2 μM. In some embodiments, the TGF-β pathway inhibitor encompasses any inhibitor that modulates or disrupts interaction of the TGF-β and its respective receptor, TGF-β receptor kinase function, or TGF-β signaling. In some instances, one or more TGF-β pathway inhibitors are included in the media composition. In some aspects, the TGF-β pathway inhibitor may be at least one of A-83-01 or DMH1.

In some embodiments of the media composition, the Wnt pathway activator may be from about 1 μM to about 10 μM in molar concentration, or any value or subrange there between. For product concentrations, the Wnt pathway activator may be included at a molar concentration of about 1.25 μM, 1.5 μM, 1.75 μM, 2.0 μM, 2.25 μM, 2.5 μM, 2.75 μM, 3.0 μM, 3.25 μM, 3.5 μM, 3.75 μM, 4.0 μM, 4.25 μM, 4.5 μM, 4.75 μM, 5.0 μM, 5.25 μM, 5.5 μM, 5.75 μM, 6.0 μM, 6.25 μM, 6.5 μM, 6.75 μM, 7.0 μM, 7.25 μM, 7.5 μM, 7.75 μM, 8.0 μM, 8.25 μM, 8.5 μM, 8.75 μM, 9.0 μM, 9.25 μM, 9.5 μM, 9.75 μM, or 10.0 μM. In some instances, the Wnt pathway activator is included at a molar concentration of about 2 μM. In some embodiments, the Wnt pathway activator encompasses any activator of Wnt signaling or activator of the Wnt/β-catenin pathway. In certain embodiments, the Wnt pathway activator may be CHIR99021.

In some embodiments of the media composition, the ROCK inhibitor may be from about 1 μM to about 10 μM in molar concentration, or any value or subrange there between. For product concentrations, the ROCK inhibitor further may be included at a molar concentration of about 1.25 μM, 1.5 μM, 1.75 μM, 2.0 μM, 2.25 μM, 2.5 μM, 2.75 μM, 3.0 μM, 3.25 μM, 3.5 μM, 3.75 μM, 4.0 μM, 4.25 μM, 4.5 μM, 4.75 μM, 5.0 μM, 5.25 μM, 5.5 μM, 5.75 μM, 6.0 μM, 6.25 μM, 6.5 μM, 6.75 μM, 7.0 μM, 7.25 μM, 7.5 μM, 7.75 μM, 8.0 μM, 8.25 μM, 8.5 μM, 8.75 μM, 9.0 μM, 9.25 μM, 9.5 μM, 9.75 μM, or 10.0 μM. In some instances, the ROCK inhibitor is included at a molar concentration of about 10 μM. In some instances, the ROCK inhibitor is a ROCK1 inhibitor. In other instances, the ROCK inhibitor is a ROCK2 inhibitor. In some cases, the ROCK inhibitor may be Y27632. In some cases, the ROCK inhibitor may be fasudil.

In some embodiments of the media composition, the EGF may range from about 25 ng/mL to about 200 ng/mL, or any value or subrange there between. In some instances, the EGF is included at a concentration of about 50 ng/mL.

In some embodiments of the media composition, the fetal bovine serum (FBS) may be from about 1% to about 10% volume concentration (v/v), or any value or subrange there between. For certain product concentrations, the fetal bovine serum may be included at a volume concentration (v/v) of about 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, or 10.0%. In some instances, FBS is included at a concentration of about 5%.

In some embodiments of the media composition, the keratinocyte growth factor (KGF) may be from about 25 ng/mL to about 200 ng/mL, or any value or subrange there between. For product concentrations, KGF further may be included at a concentration of about 25 ng/mL, 30 ng/mL, 40 ng/mL, 50 ng/mL, 60 ng/mL, 70 ng/mL, 80 ng/mL, 90 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, or 200 ng/mL. In some cases, KGF is included at a concentration of from about 50 ng/mL to about 100 ng/mL.

In some embodiments of the media composition, the composition comprises a basal medium which is further supplemented with one or more additional components such as a TGF-β pathway inhibitor, a Wnt pathway activator, a ROCK inhibitor, an epidermal growth factor (EGF), a keratinocyte growth factor (KGF), a fetal bovine serum (FBS), and optionally amino acids such as L-glutamine and/or an antibiotic. In some instances, the basal medium is DMEM/F-12 medium. In some instances, the composition comprises L-glutamine (e.g., GlutaMAX™). In some cases, the media composition comprises from about 50 μg/mL to about 200 μg/mL, optionally from about 100 μg/mL to about 200 μg/mL or from about 100 μg/mL to about 150 μg/mL of the antibiotic. In some cases, the media composition comprises about 100 μg/mL of the antibiotic. In some instances, the antibiotic is Primocin™.

In some embodiments, the media composition comprises a basal medium selected from DMEM/F-12 medium. In some cases, the media composition further comprises about 2.5 mM L-glutamine, about 5% FBS, about 2 μM of a first TGF-β pathway inhibitor, about 1 μM of a second TGF-β pathway inhibitor, about 2 μM of a Wnt pathway activator, about 50 ng/mL of EGF, about 50-100 ng/mL of KGF, about 10 μM of a ROCK inhibitor, and about 100 μg/mL of an antibiotic.

In some embodiments, the media composition comprises a basal medium selected from DMEM/F-12 medium. In some cases, the media composition further comprises 2.5 mM concentration of GlutaMAX™, about 5% FBS, about 2 μM of A-83-01, about 1 μM of DHM1, about 2 μM of CHIR99021, about 50 ng/mL of EGF, about 50-100 ng/mL of KGF, about 10 μM of Y27632, and about 100 μg/mL of Primocin™. In some cases, this media composition is also referred to herein as O-WREKT media.

The composition for culturing alveolar epithelial cells further may include a plurality of three-dimensional substrates. In some embodiments, the three-dimensional substrates can be a plurality of at least one of a solid, microporous, or macroporous three-dimensional substrates. In certain embodiments, the microporous three-dimensional substrates further comprise microcarriers.

### Kits and Articles of Manufacture

In some embodiments, a kit or article of manufacture described herein includes one or more populations of the alveolar epithelial cells obtained by a method described supra or one or more populations of alveolar epithelial cells cultured in a cell culture media composition described supra. In some instances, the kit or article of manufacture described herein further include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

### EXAMPLES

These examples are provided for illustrative purposes only and not to limit the scope of the claims provided herein.

### Example 1

Parameter Optimization

Microcarrier information: the following microcarriers were tested for AT2 cell attachment. The microcarriers were: 1) Percell CultiSpher S microcarriers, 2) Percell CultiSpher GL microcarriers, 3) Corning collagen dissolvable microcarriers, 4) Corning SyntheMax II dissolvable microcarriers, 5) Percell CultiSpher G microcarriers, and 6) GE Cytodex3 microcarriers. AT2 cells attached to all microcarriers, but attachment was most prominent on CultiSpher GL and Corning collagen dissolvable microcarriers, based on LIVE/DEAD™ imaging of AT2 cells after 24 hours.

Media Optimization: Different cell culture media were tested to determine optimal growth conditions. In one assay, three separate cell culture media were tested: 1) Gibco DMEM F-12 media with a TGF-β pathway inhibitor; a Wnt pathway activator; a ROCK inhibitor; an epidermal growth factor (EGF); a keratinocyte growth factor (KGF); and a fetal bovine serum (FBS), 2) Lonza SAGM media, and 3) Stemcell Technologies Pneumacult ALI media. The media described in 1) outperformed other growth media tested.

Seeding Optimization: Intermittent and continuous agitation seeding was tested to improve attachment efficiency of AT2 cells on microcarriers. Intermittent agitation was found to improve AT2 attachment, as determined by live/dead staining at 18 hrs.

Protocol

Microcarrier preparation: First, the microcarriers were prepared before seeding. Microcarrier preparation included adding 0.2 grams Percell CultiSpher GL microcarriers to a 250 mL spinner flask. The microcarriers were then hydrated for at least one hour (ranged from 1 hour to overnight) in 100-150 mL Dulbecco's phosphate-buffered saline (DPBS) and autoclaved in spinner flasks to sterilize. DPBS was aspirated and the microcarriers were then washed with DPBS. DPBS was then exchanged for 100 mL cell culture media. The spinner flask was then moved to 37° C., 5% CO₂ and incubated. Agitation was performed on a stir plate in the incubator at 32 RPM for about 2 hours to allow for equilibration. After seeding, culture media may be added to reach full culture volume.

AT2 cell seeding: In one example, after equilibration, 5,000-10,000 AT2 cells/cm² were added in cell culture media to each spinner flask, and seeded using intermittent agitation for 18 hours. Agitation occurred in the following intervals: Time ON: 5 minutes; Agitation: 32 RPM; Time OFF: 30 minutes. In this embodiment of the exemplary method, about 31 cycles were completed. After approximately 18 hours, continuous agitation began at about 32 RPM for the remainder of the culture.

Culture Feeding: The culture was fed about every two to about every four days, and metabolic samples were taken daily and after feeds. Feeding took place in a biosafety cabinet, after sufficient time had passed to allow the microcarriers to settle.

Culture Monitoring: An about 2 mL media sample was taken using a syringe and run on a NOVA Flex 2 bioanalyzer. The sample was analyzed for at least pH, glucose, lactate, glutamine, and ammonium levels. The spinner flask was then returned to the stir plate.

Samples were then taken and a LIVE/DEAD™ assay was performed about every two to about every three days throughout the culture. In a biosafety cabinet, the spinner flask was swirled to suspend the microcarriers. An about 0.5 mL sample was taken of the microcarriers and the media and added to a microcentrifuge tube. Microcarriers were then stained according to the manufacturers' protocol (ThermoFisher Live/Dead™ Cell Imaging Kit, Product #R37601) and imaged to assess cell density and coverage.

Harvesting alveolar epithelial cells: In this exemplary embodiment of the claimed methods, AT2 cells were harvested from the microcarriers. Harvest day was based on a confluency assessment of satellite 2D culture of cells as well as a Live/Dead imaging assay (typically about 13 to about 16 days in culture). Microcarriers were allowed to settle, and almost all media was aspirated from each spinner. Spinner flasks were washed twice with 200 mL DPBS, aspirating almost all DPBS each time. About 150 mL of about 0.25% trypsin was then added to each spinner. Spinners were then returned to incubators and agitated for about 15-20 minutes at about 32 RPM. Cell solution was then collected into about 50 mL centrifuge tubes, and each spinner was rinsed with about 50 mL DPBS with 2% fetal bovine serum (FBS). Rinse was then collected in centrifuge tubes. Cells were spun down at about 300×g for about 15 minutes. Supernatant was then aspirated and each sample was resuspended in 20 mL DPBS and cells were counted

If continuing the culture over a next passage post-harvest, then each step of this protocol may be performed again in continued passaging.

Results

In the disclosed protocol, AT2 cells were cultured through three passages and the feasibility of culturing AT2 cells on microcarriers in an about 250 mL glass spinner flask was demonstrated. As shown in FIG. 1, an increase in the number of AT2 cells on microcarriers was visibly evident for all three passages, indicating successful attachment and growth of AT2 cells on microcarriers.

As illustrated graphically in the top panel of FIG. 2, HT2-280 expression—a marker used to identify AT2 cells—was better maintained in three-dimensional culture on microcarriers compared to standard two-dimensional culture across both trials. FIG. 2 also shows that overgrowth of contaminating airway basal cells (CK5+) was slowed for AT2 cell cultures on microcarriers in both attempted trials, which has been one of the biggest hurdles for AT2 expansion to date. AT2 cells cultured in two-dimensional culture were overgrown by airway basal cells within one to two passages.

FIG. 3 indicates that AT2 function was better maintained on microcarriers than in standard two-dimensional culture, as evidenced by better maintenance of SP-C expression in the first passage of culture. These results are compared with standard two-dimensional culture, where SP-C expression is typically lost within the first few days of culture.

### Example 2

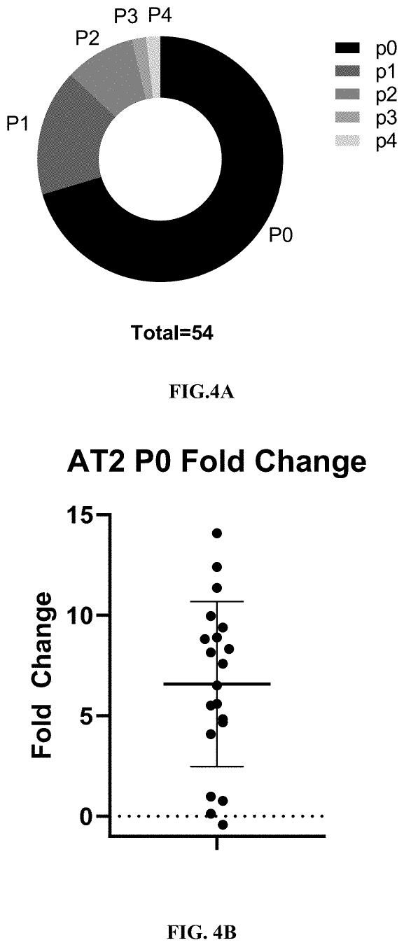

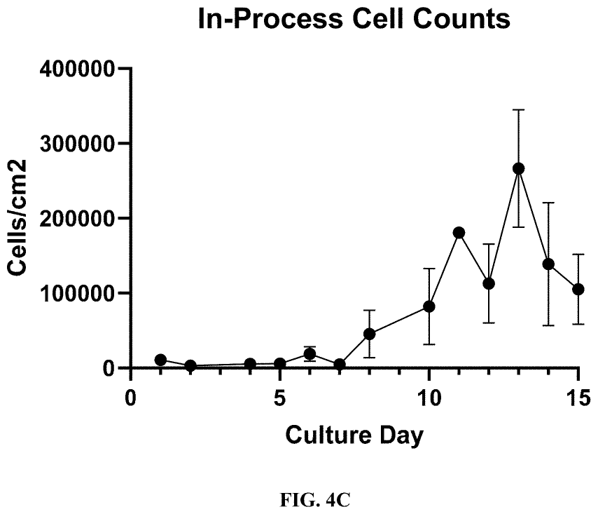

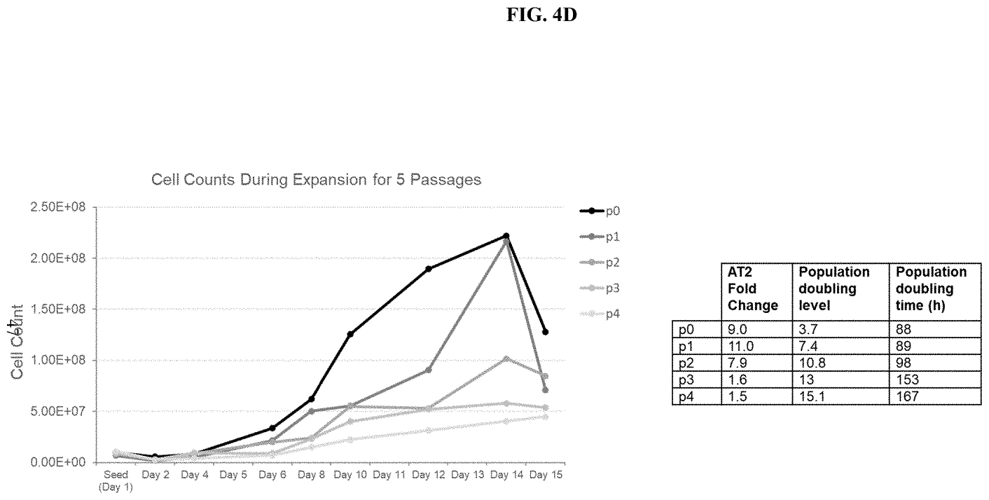

AT2 cell expansions were carried out in over 50 runs in 250 mL spinner flasks. Cells from 11 different lung donors were tested. FIG. 4A illustrates a summary of all spinner flask AT2 microcarrier expansions performed in 250 mL spinner flasks, broken down by passage. Equal numbers of runs were performed from freshly isolated cells and previously frozen cells. FIG. 4B-4C illustrate AT2 fold change (mean 6.6) and average in-process cell counts of 250 mL spinner flask passage 0 expansion runs performed with Percell Cultispher GL microcarriers in the O-WREKT media. FIG. 4D depicts in-process cell counts and expansion characteristics (AT2 fold change, population doubling level, and population doubling time) from a 250 mL spinner flask expansion carried out for 5 passages (passage 0-passage 4) on Percell Cultispher GL microcarriers in the O-WREKT media. This data demonstrates substantial growth for 3 passages from passage 0 to passage 3. As described above, the O-WREKT media comprises DMEM/F-12 medium, 2.5 mM concentration of GlutaMAX™, about 5% FBS, about 2 μM of A-83-01, about 1 μM of DHM1, about 2 μM of CHIR99021, about 50 ng/mL of EGF, about 50-100 ng/mL of KGF, about 10 μM of Y27632, and about 100 μg/mL of Primocin™.

### Example 3

FIG. 5 illustrates an exemplary bioreactor expansion process. In some embodiments, AT2 cells were isolated and purified from human donor lung tissue. Next, the microcarriers were prepared for cell seeding. Microcarrier preparation included adding 10 grams Percell CultiSpher GL microcarriers to a bottle. The microcarriers were then hydrated for at least one hour (ranged from 1 hour to overnight) in 2-3 L Dulbecco's phosphate-buffered saline (DPBS) and autoclaved to be sterilized. DPBS was removed and then exchanged for 4.5 L cell culture media. Microcarriers were transferred to the bioreactor. Agitation was performed at about 40 RPM for about 2 hours to allow for equilibration.

AT2 cell seeding: In one example, after equilibration, 550e⁶ AT2 cells were added in cell culture media to a bioreactor and seeded using intermittent agitation for 18 hours. Agitation occurred in the following intervals: Time ON: 5 minutes; Agitation: 42 RPM; Time OFF: 30 minutes. In this embodiment of the exemplary method, about 32 cycles were completed. After approximately 18 hours, continuous agitation began at about 39 RPM for the remainder of the culture. After seeding, culture media may be added to reach full culture volume.

Culture Feeding: The culture was fed about every two days, and metabolic samples were taken daily and after feeds. Feeding took place after sufficient time had passed to allow the microcarriers to settle.

Culture Monitoring: In-process counts and probe for measuring one or more of pH, glucose, lactate, glutamine, ammonium, or dissolved oxygen levels or biocapacitance was utilized.

Harvesting alveolar epithelial cells: In this exemplary embodiment of the claimed methods, AT2 cells were harvested from the microcarriers. Microcarriers were allowed to settle, and almost all media was removed from the bioreactor. The microcarriers were washed once with 5 L DPBS, removing almost all DPBS. About 3-4 L of about 0.25% trypsin was then added to the bioreactor, and then agitated for about 15-45 minutes at about 39 RPM. Cell solution was then collected into a sterile bioprocess container. The bioreactor was rinsed with about 1 L DPBS with 5% fetal bovine serum (FBS). Rinse was then collected in the same bioprocess container and subsequently transferred to centrifuge tubes. Cells were spun down at about 300×g for about 15 minutes. Supernatant was then aspirated and each sample was resuspended in about 1 L DPBS and cells were counted.

If continuing the culture over a next passage post-harvest, then each step of this protocol may be performed again in continued passaging.

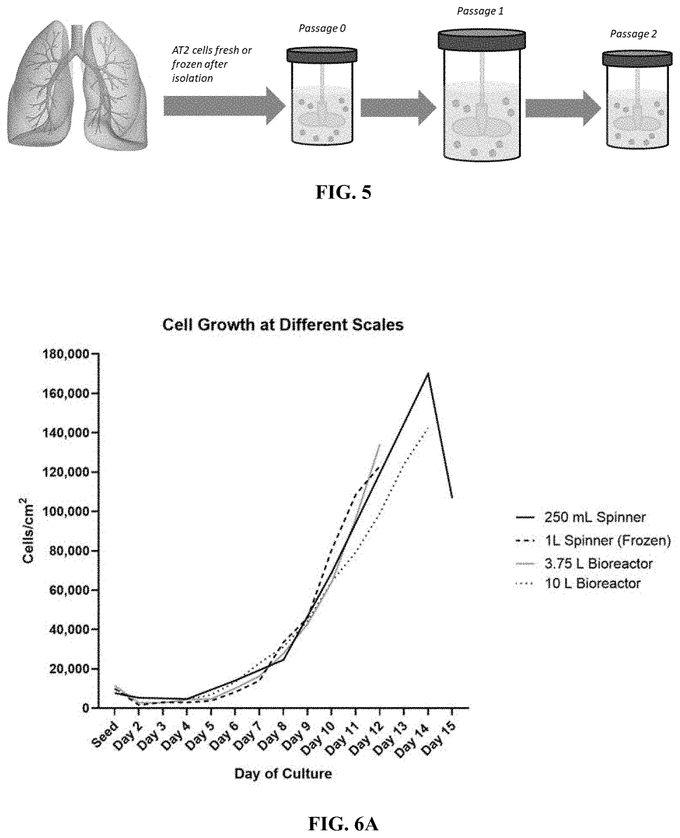

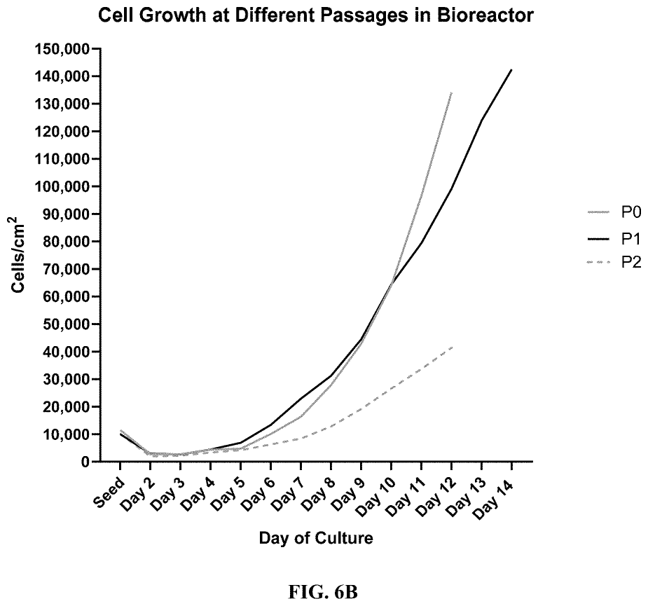

FIG. 6A and FIG. 6B illustrate growth profiles at different scales (spinner flask and bioreactor) and passages in a bioreactor.

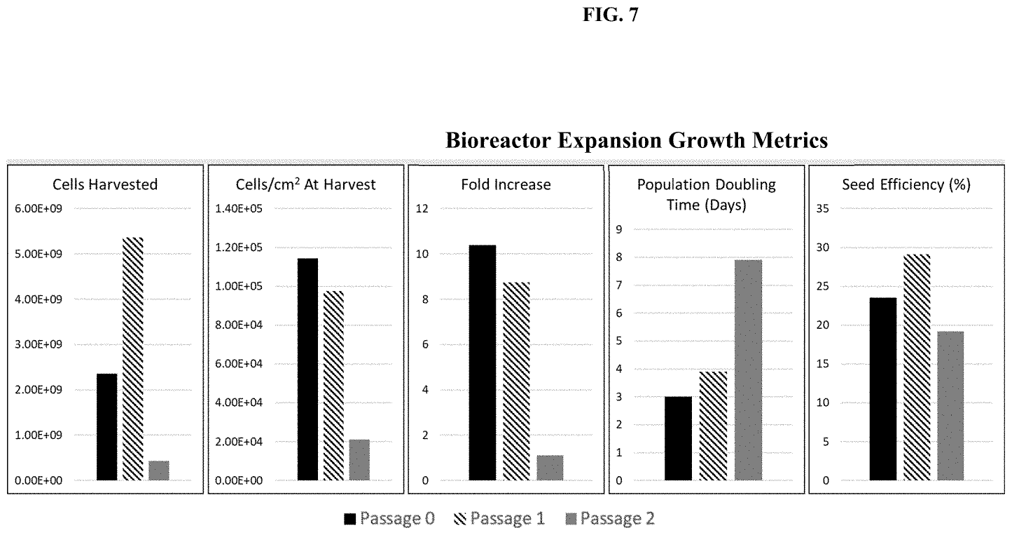

FIG. 7 illustrates bioreactor expansion growth metrics from passage 0 to passage 2.

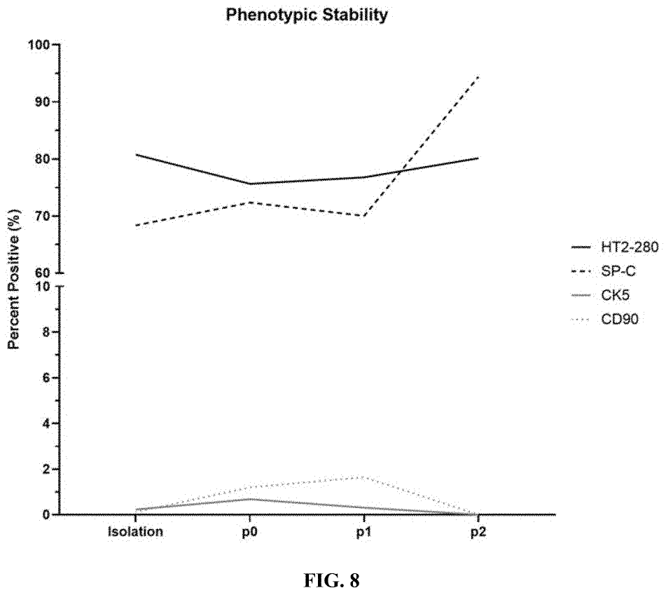

FIG. 8 illustrates bioreactor expansion phenotypic analysis. HT2-280 and SP-C were maintained for 3 passages. CK5+ basal cell and CD90+ stromal cell overgrowth were not observed.

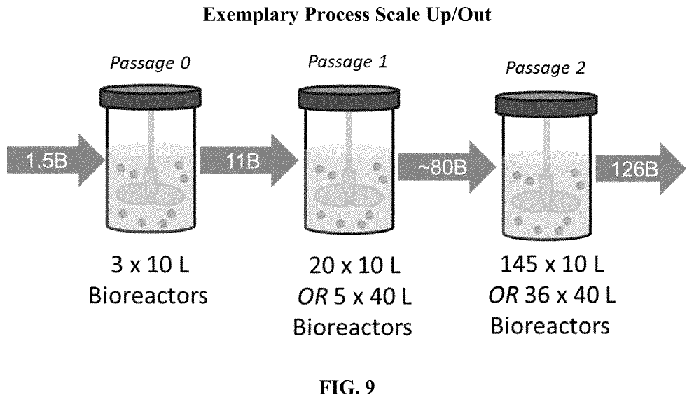

FIG. 9 illustrates exemplary process scale up/out for use with one or more of the methods described herein.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A method for producing alveolar epithelial cells, comprising:
preparing a plurality of three-dimensional substrates in a cell culture vessel;
seeding a plurality of alveolar epithelial cells, wherein seeding comprises combining the three-dimensional substrate and the alveolar epithelial cells in the cell culture vessel and providing conditions suitable to enable attachment of the cells to the three-dimensional substrate to create a suspension culture;
promoting growth of the alveolar epithelial cells on or within the three-dimensional substrates;
monitoring the culture for cell proliferation; and
harvesting a plurality of alveolar epithelial cells from the three-dimensional substrates.

2. The method of claim 1, wherein the alveolar epithelial cells comprise alveolar type II epithelial (AT2) cells.

3. The method of claim 1, wherein the alveolar epithelial cells comprise human alveolar type II epithelial cells (AT2).

4. The method of claim 1, wherein the three-dimensional substrates comprise at least one of a solid, microporous, or macroporous three-dimensional substrates.

5. The method of claim 4, wherein the alveolar epithelial cells are cultured on top of or within or both on top of and within the three-dimensional substrates.

6. The method of claim 4, wherein the three-dimensional substrate comprises a plurality of microcarriers.

7. The method of claim 1, wherein the cell culture vessel comprises a spinner flask or bioreactor.

8. The method of claim 1, wherein the three-dimensional substrates comprise about 1-2 mg/mL.

9. The method of claim 1, wherein seeding further comprises agitating the culture.

10. The method of claim 9, wherein the three-dimensional substrate culture is agitated in the cell culture vessel at about 20 RPM or higher.

11. The method of claim 9, wherein the agitation comprises a cycle wherein agitation occurs for about 5 minutes at about 20 RPM or higher followed by about 30 minutes of no agitation, further wherein the cycle is repeated about 31 times.

12. The method of claim 11, wherein after about 18 hours the culture is then agitated at about 20 RPM or higher continuously for the remainder of the culture.

13. The method of claim 1, wherein monitoring comprises feeding the culture, performing at least one live/dead assay on the culture, assessing a measurement of pH, glucose, lactate, glutamine, ammonium, and/or dissolved oxygen levels and/or biocapacitance, assessing cell coverage on the three-dimensional substrates, or a combination thereof.

14. The method of claim 13, wherein the culture is fed at intervals of about two days to about four days and a metabolic sample is taken daily and/or after a feed.

15. The method of claim 1, wherein harvesting a plurality of alveolar epithelial cells from the three-dimensional substrates further comprises allowing the three-dimensional substrates to settle and removing a quantity of media from the cell culture vessel, washing the cell culture vessel, adding a quantity of an agent to detach the cells from the three-dimensional substrates, agitating the cell culture vessel, collecting a cell solution into centrifuge tubes, rinsing the cell culture vessel, collecting a quantity of rinse from the cell culture vessel, spinning the quantity of rinse, aspirating the supernatant of the rinse, and resuspending any sample in phosphate buffered saline, further wherein harvesting is performed between about 10 to about 18 days of culture.

16. The method of claim 15, wherein the harvested cells are seeded onto new three-dimensional substrates and continued in culture or are cryopreserved.

17. The method of claim 1, wherein the plurality of harvested alveolar epithelial cells:
express pro-surfactant protein C (pSP-C);
typically lose no more than 25% of pSP-C expression in up to and about 40 days of culture;
comprise a population with pSP-C expression greater than about 30% after up to and about 40 days;
express HT2-280;
does not express an excess of CK5 or comprise an overgrowth of airway basal cells; or a combination thereof.

18. The method of claim 1, wherein the alveolar epithelial cells are cultured for one or more passages.

19. The method of claim 6, wherein the microcarriers comprise a stiffness between about 1 kPa to about 100 kPa.

20. A cell culture media composition for culturing alveolar epithelial cells comprising:
a TGF-β pathway inhibitor;
a Wnt pathway activator;
a ROCK inhibitor;
an epidermal growth factor (EGF);
a keratinocyte growth factor (KGF); and
a fetal bovine serum.

21. The composition of claim 20, wherein the TGF-β pathway inhibitor comprises about 1 μM to about 10 μM.

22. The composition of claim 20, wherein the Wnt pathway activator comprises about 1 μM to about 10 μM.

23. The composition of claim 20, wherein the ROCK inhibitor comprises about 1 μM to about 10 μM.

24. The composition of claim 20, wherein the EGF comprises between about 25 ng/mL to about 200 ng/mL.

25. The composition of claim 20, the KGF comprises between about 25 ng/mL to about 200 ng/mL.

26. The composition of claim 20, wherein fetal bovine serum comprises about 1% to about 10% volume concentration (v/v).

27. The composition of claim 20, wherein TGF-β inhibitor comprises at least one of A-83-01 or DMH1.

28. The composition of claim 20, wherein Wnt pathway activator comprises CHIR99021.

29. The composition of claim 20, wherein the ROCK inhibitor comprises Y27632.

30. The composition of claim 20, wherein the composition further comprises a plurality of three-dimensional substrates.
